(19) United States
(12) Patent Application Publication
(10) Pub. No.: US 2012/0040392 A1
(43) Pub. Date: Feb. 16, 2012

(54) DOSIMETRY USING SIGMA SINGLET OXYGEN SPECTROSCOPY

(75) Inventor: Sung-Wei Chen, Singapore (SG)

(73) Assignee: Empire Technology Development LLC

(21) Appl. No.: 13/120,714

(22) PCT Filed: Aug. 16, 2010
(86) PCT No.: PCT/SG10/00301

§ 371 Date: Mar. 24, 2011

(51) Int. Cl. C12Q 1/02; C12M 1/34

(52) U.S. Cl. 435/29; 435/288.7

(57) ABSTRACT

A method includes measuring a photoluminescence of sigma singlet state oxygen decaying to triplet state oxygen. A dosage of delta singlet state oxygen is determined based on the photoluminescence.

### BACKGROUND

The following description is provided to assist the understanding of the reader. None of the information provided or references cited is admitted to be prior art.

In photodynamic therapy (PDT), photosensitizer compounds absorb light and transfer the energy through intermediates (Type I reaction) or directly transfer the energy to triplet ground-state molecular oxygen (Type II reaction) producing singlet oxygen as a therapeutic agent. This process is analogous to fluorescence except that the outgoing energy is transferred directly to triplet oxygen.

Dosimetry is the measurement of therapeutic efficacy and dose. Dosimetry in Type II photosensitizers can be difficult for PDT. PDT efficacy is highly sensitive to the various dose components in vivo. Henderson, B. W., et al. “Fluence Rate as a Modulator of PDT Mechanisms,” Lasers in Surgery and Medicine (2006) 28:489-493. Currently, delta singlet oxygen can be monitored in PDT using chemilphotoluminescence with an added agent. Wei, Y., et al. “In vivo Monitoring of Singlet Oxygen Using Delayed Chemiphotoluminescence During Photodynamic Therapy,” Journal of Biomedical Optics (2007) 12: 014002.

### SUMMARY

In one aspect, a method includes measuring a photoluminescence of sigma singlet state oxygen decaying to triplet state oxygen. A dosage of delta singlet state oxygen can be determined based on the photoluminescence.

In one aspect, an apparatus includes a detector and a module. The detector can be configured to measure a photoluminescence of sigma singlet state oxygen decaying to triplet state oxygen. The module can be configured to determine a dosage of delta singlet state oxygen based on the photoluminescence.

In one aspect, an article of manufacture includes a computer-readable medium having instructions stored thereon that, if executed by a computing device, cause the computing device to perform operations including measuring, with a detector, a photoluminescence of sigma singlet state oxygen decaying to triplet state oxygen. A dosage of delta singlet state oxygen can be determined based on the photoluminescence.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1 is a diagram of singlet oxygen formation in accordance with an illustrative embodiment.

FIG. 2 is a diagram of oxygen molecule energy levels in accordance with an illustrative embodiment.

FIG. 3 is a graph of photoluminescence levels in accordance with an illustrative embodiment.

FIG. 4 is a schematic of a delta singlet state oxygen dosimetry system in accordance with an illustrative embodiment.

FIG. 5 is a flow diagram illustrating delta singlet state oxygen dosimetry operations performed in accordance with an illustrative embodiment.

FIG. 6 is a flow diagram of analysis software of FIG. 4 in accordance with an illustrative embodiment.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

Described herein are illustrative systems, methods, computer-readable media, etc. for dosimetry using sigma singlet oxygen spectroscopy (i.e., the energy transitions to and from sigma singlet oxygen). Dosimetry in PDT can be determined by measurement of delta singlet oxygen, which is the therapeutic moiety, in vivo. The use of photoluminescence detection or spectroscopy of the phosphorescence (approximately 1.63 eV) of sigma singlet oxygen (second excited state of molecular oxygen) in vivo can be used to measure the concentration of delta singlet oxygen. Delta singlet oxygen produced by photosensitization can be energetically upconverted into sigma singlet oxygen by addition of light energy (approximately 0.65 eV) distinct from the phosphorescence energy. Since delta singlet oxygen is the therapeutic moiety, i.e., the active agent, in photodynamic therapy, the phosphorescence of sigma singlet oxygen provides an measure of therapeutic dosing of the delta singlet oxygen.

Delta singlet oxygen can be excited to sigma singlet oxygen in vivo by an excitation input including mid-infrared radiation near 0.65 eV (i.e., approximately 1907 nm). The mid-infrared radiation can be produced by tungsten light sources, mid-infrared gallium antimonide (GaSb) heterostructure light emitting diodes (LEDs), group III-V indium gallium arsenide phosphide (InGaAsP) or gallium indium arsenide antimonide (GaInAsSb) laser diodes, or other “green field” mid-infrared (mid-IR) sources. The sigma singlet oxygen can decay to the ground state and emit visible red light near 1.63 eV (i.e., approximately 762 nm). The visible red light photoluminescence from the sigma singlet oxygen can be detected by a charge-coupled device (CCD) camera, cooled bolometric detectors, or any other quantitative photon detection device.

The delta singlet oxygen concentration can be measured in vivo to determine dosimetry in a photodynamic therapy. The excitation wavelength and detection wavelength can be adjusted to account for red shifting of wavelengths by biological media. Red shifting of light within biological material is generally the result of the absorption of photonic energy when light is scattered and refracted in the biological material. Generally, observed and transmitted photon wavelengths can be adjusted to account for the red shift effects of the particular biological material. The dosimetry can be determined using curve fitting methods, analyses of exponential decay of photoluminescence, Fourier transform spectroscopy, and other time resolved methods as described further below. In the following illustrative embodiments, though precise energy values are generally used, a practical implementation can excite or detect within a bandwidth given the red shift (e.g. +/−5 nm) in a given biological matter.

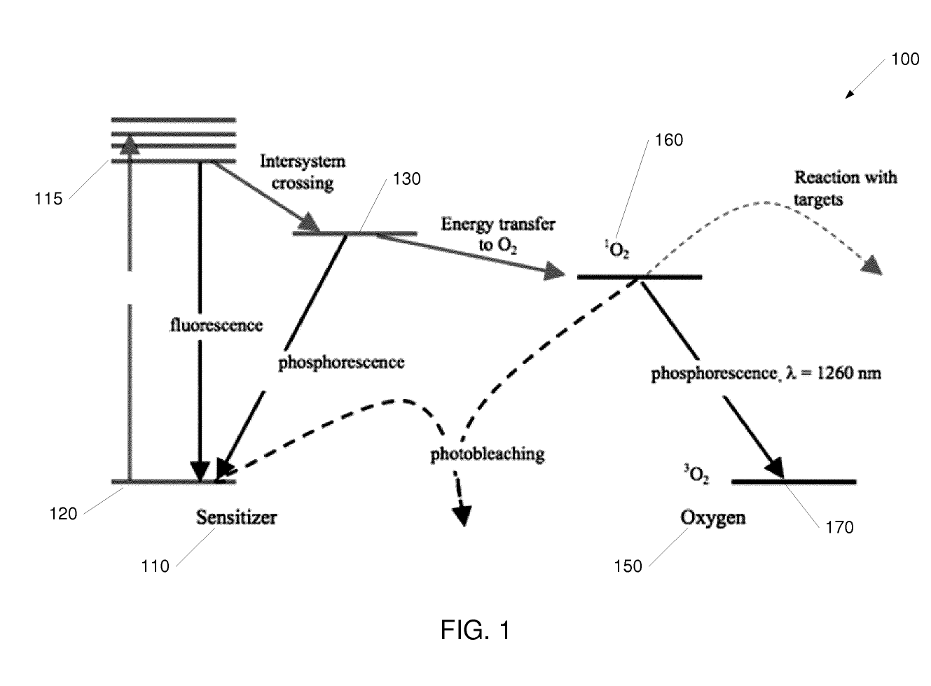

Referring to FIG. 1, a diagram of singlet oxygen formation in accordance with an illustrative embodiment is shown. In particular, FIG. 1 depicts a Jablonski diagram 100 of singlet oxygen formation by a Type II photosensitizer. Solid lines are monomolecular transitions and dashed lines are bimolecular reactions. The Jablonski diagram 100 illustrates the electronic states of a photosensitizer 110 (represented by the states on the left hand of the Jablonski diagram 100) and an oxygen molecule 150 (represented by the states on the right hand of the Jablonski diagram 100), and the transitions between them.

The photosensitizer 110 is a chemical compound that can be excited by light of a specific wavelength, examples of which are described below. This excitation can involve visible or near-infrared light. In photodynamic therapy, either a photosensitizer or the metabolic precursor of one can be administered to a patient. A tissue of the patient to be treated is exposed to light suitable for exciting the photosensitizer. The photosensitizer 110 can be, for example, but not limited to, porfimer sodium, aminolevulinic acid, methyl aminolevulinate, porphyrins, silicon phthalocyanine, m-tetrahydroxyphenylchlorin, or mono-L-aspartyl chlorine.

Photosensitizers are generally excited by 600 nm-800 nm light; however, photosensitizers that can be excited by any wavelength can be used. Examples of photosensitizers, their trade names and/or common name in parenthesis, and approximate excitation wavelength(s) include, but are not limited to: porfimer sodium (Photofrin) 630 nm, aminolevulinic acid (Levulan) 630-635 nm, methyl aminolevulinate (Metvixia) 570-670 nm, porphyrins 570-670 nm, silicon phthalocyanine and its derivates 672 nm, m-tetrahydroxyphenylchlorin (Foscan) 514 nm and 652 nm, and mono-L-aspartyl chlorine (Laserphyrin, talaporfin, NPe6, LS11, aspartyl chlorin) 664 nm. Some photosensitizers can have multiple excitation peaks. In addition, the excitation wavelength can be selected to produce a non-optimal effect.

The photosensitizer 110 is excited from a ground photosensitizer state 120 to an excited photosensitizer state 115. The photosensitizer 110 can then undergo intersystem crossing to a longer-lived excited photosensitizer triplet state 130. The photosensitizer 110 can fluoresce from the excited photosensitizer state 115 to the ground photosensitizer state 120.

When the photosensitizer 110 and the oxygen molecule 150 are in proximity, an energy transfer can take place that allows the photosensitizer to relax to the ground photosensitizer state 120, and create a singlet state oxygen molecule 160. For example, after creating the singlet state oxygen molecule 160, the photosensitizer 110 phosphoresces to the ground photosensitizer state 120. The singlet state oxygen molecule 160 can be a delta singlet state oxygen molecule or a sigma singlet state oxygen molecule.

Singlet oxygen is an aggressive chemical species and can very rapidly react with targets, for example, any nearby biomolecules, and/or cells. In photodynamic therapy, the singlet state oxygen molecule 160 can damage cell membranes, nucleic acids, and/or proteins, etc. which may lead to cell death by apoptosis. The singlet state oxygen molecule 160 can phosphoresce (e.g., emitting a 1260 nm photon) back to a triplet state oxygen molecule 170 (i.e., the ground state).

Photobleaching can also occur involving the ground photosensitizer state 120 and the singlet state oxygen molecule 160. Photobleaching is the photochemical destruction of a fluorophore. For example, the photosensitizer 110 is destroyed.

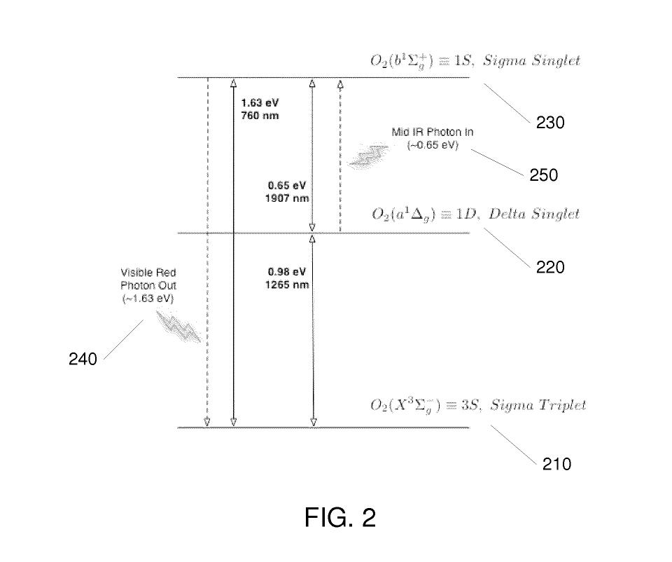

Referring to FIG. 2, a diagram of oxygen molecule energy levels in accordance with an illustrative embodiment is shown. In particular, FIG. 2 depicts the ground state and first two excited states of molecular oxygen. These energies are valid in gas phase and in solution. Red shift solvent effects move the wavelengths down in energy (i.e., longer wavelengths) as described by Lakowicz, J. R. “Principles of Fluorescence Spectroscopy,” 3rd Edition. Springer 2006, which is incorporated herein by reference in its entirety.

An oxygen molecule can exist in a sigma triplet state 210, a delta singlet state 220, and a sigma singlet state 230. The sigma triplet state 210 is the ground state. The oxygen molecule can be excited from the sigma triplet state 210 to the delta singlet state 220 when the oxygen molecule, in the sigma triplet state 210, absorbs radiation near 0.98 eV (i.e., approximately 1265 nm). The excitation of the oxygen molecule may arise, for example, from using a photosensitizer, as described above. An oxygen molecule in the delta singlet state 220 can be used in, for example, photodynamic therapy, as described above. The photosensitizer can be excited by a first light source as explained further below.

The oxygen molecule can be excited from the delta singlet state 220 to the sigma singlet state 230 when the oxygen molecule, in the delta singlet state 220, absorbs mid-infrared radiation near 0.65 eV (i.e., approximately 1907 nm). For example, the oxygen molecule can absorb a 0.65 eV photon 250. The 0.65 eV photon 250 can be provided by a second light source such as a tungsten light source, mid-infrared GaSb heterostructure LEDs, group III-V InGaAsP or antimony GaInAsSb laser diodes, or other “green field” mid-IR sources.

The oxygen molecule can decay from the sigma singlet state 230 to the sigma triplet state 210, emitting visible red light near 1.63 eV (i.e., approximately 762 nm). For example, the oxygen molecule can emit a 1.63 eV photon 240. The 1.63 eV photon 240 can be detected by a detector, for example, a CCD camera, cooled bolometric detectors, or any other quantitative photon detection device. The oxygen molecule can also decay from the sigma singlet state 230 back to the delta singlet state 220, emitting a 0.65 eV photon. The decay back to the delta singlet state 220 predominates the decay to the sigma triplet state 210 by a factor of about 3000 to 1 as discussed in Keszthelyi, T., et al. “Radiative Transitions of Singlet Oxygen: New Tools, New Techniques and New Interpretations,” Photochemistry and Photobiology (1999) 70: 531-539; and Weldon, D., et al. “Singlet Sigma: The ‘Other’ Singlet Oxygen in Solution,” Photochemistry and Photobiology (1999) 70: 369-379, both of which are incorporated herein by reference in their entirety. However, the kinetics of the decay from the sigma singlet state 230 to the sigma triplet state 210 are easier to observe.

The 1.63 eV photons are relativity easy to detect since they lie in the 762 nm range of visible red light (and may be red shifted down even more by the medium). Commercially available CCD-based and other detectors can be used for detection. 0.65 eV photons, on the other hand, require mid-IR detectors, which are relatively slow and expensive. In addition, detection of the 0.65 eV photons can be complicated by background auto-fluorescence (e.g., from the photosensitizer) and the excitation source (i.e., the light source used to excite the photo sensitizer).

Advantageously, the concentration of delta singlet state 220 oxygen molecules can be measured by exciting oxygen molecules in the delta singlet state 220 to the sigma singlet state 230 and then observing the intensity of the emission from the sigma singlet state 230 to the sigma triplet state 210. The photoluminescence kinetics of the emission from the sigma singlet state 230 to the sigma triplet state 210 then enables deduction of the delta singlet state 220 oxygen molecule concentration, as explained further below. In an illustrative embodiment, in PDT, the dosimetry of delta singlet state oxygen can be determined and controlled, as explained further below. Advantageously, the determination of delta singlet state 220 oxygen molecule concentration can be performed with simple, economical, and readily available excitation and detection equipment.

Other illustrative applications include, combustion monitoring, military/chemical sensing, semiconductor processing, industrial sensing, and automotive sensing. The concentration of delta singlet state oxygen molecules can be determined in any process that uses or produces oxygen. In one illustrative example, the concentration of endogenously produced delta singlet state oxygen molecules can be determined. For example, during combustion, a population of delta singlet oxygen can be produced by combustion reactions. The endogenously produced delta singlet state oxygen molecules can be exogenously excited to a sigma singlet state, as discussed above. The kinetics of the process that uses or produces oxygen (e.g., combustion) can be used to calculate various oxygen populations, as discussed further below. Additional examples, of illustrative processes include, but are not limited to, military applications such as monitoring oxygen in confined spaces or diving equipment, semiconductor processing applications such as oxidization monitoring or plasma etch monitoring, and automotive applications such as pollution monitoring. The concentration of delta singlet state oxygen molecules can be used, for example, to detect and monitor spectroscopic sensitivity, signal-to-noise ratio, or other detection reasons in a process.

The concentration of delta singlet state oxygen molecules in an object can be determined by a kinetic differential equation. The concentration of delta singlet state oxygen molecules in a 0.65 eV excitation system can be determined, for example, using Equation 1, below. The concentration of delta singlet state oxygen molecules (i.e., the first term of Equation 1) is proportional to the radiative transitions from the sigma singlet state to the sigma triplet state and delta singlet state, respectively (i.e., the third and fourth terms of Equation 1) as discussed in Niedre, M., et al. “Direct Near-Infrared Photoluminescence Detection of Singlet Oxygen Generated by Photodynamic Therapy in Cells In Vitro and Tissues In vivo,” Photochemistry and Photobiology (2002) 75: 382-391; and Patterson, M. S., et al. “Experimental Tests of the Feasibility of Singlet Oxygen Photoluminescence Monitoring in vivo During Photodynamic Therapy,” Journal of Photochemistry and Photobiology, B: Biology (1990) 5: 69-84, both of which are incorporated herein by reference in their entirety. Equation 1 can be simplified further, for example, in the steady state and where the form of the input energy is known.

$$\begin{matrix}\frac{d [ {}_{}^{1}{\sum }{}_{g}^{+} ]}{d t} = {I}_{\sum } {σ}_{Δ} {[}^{1} {O}_{2} ] - \frac{{[}^{1} {\sum }_{g}^{+} ]}{{τ}_{1.63}} - \frac{{[}^{1} {\sum }_{g}^{+} ]}{{τ}_{0.65}} & ( 1 ) \\ \begin{matrix}{[}^{1} {O}_{2} ] = \frac{1}{{I}_{\sum } {σ}_{Δ}} {[}^{1} {\sum }_{g}^{+} ] ( \frac{1}{{τ}_{1.63}} + \frac{1}{{τ}_{0.65}} ) & ( in steady state )\end{matrix} & ( 2 ) \\ \begin{matrix}{[}^{1} {O}_{2} ] = \frac{1}{N {σ}_{Δ} \cdot exp ( \frac{- t}{{τ}_{1.63}} + \frac{- t}{{τ}_{0.65}} )} {[}^{1} {\sum }_{g}^{+} ] & ( with {I}_{\sum } = N \cdot δ ( t ) )\end{matrix} & ( 3 )\end{matrix}$$

Definitions:

[¹Σ_{g}⁺]=concentration of sigma singlet (¹Σ_{g}⁺, 1S), in molecules/cm³

[¹O₂]=concentration of delta singlet (¹Δ, 1D), in molecules/cm³

I_{Σ}=photon fluence, in W/cm²

σ_{Δ}=cross section of delta singlet for excitation to sigma singlet, in cm⁻²

τ_{1.63}=lifetime ³Σ_{g}⁻→¹Σ_{g}⁺ (3S→1S) transition (1.63 eV spacing), in s⁻¹

τ_{0.65}=lifetime ¹Δ→¹Σ_{g}⁺ (1D→1S) transition (0.65 eV spacing), in s⁻¹

N=photons in the δ-function impulse, in # of photons

Equation 2 is a simplification of Equation 1 where the production of delta singlet state oxygen molecules is in the steady state and the delta singlet state oxygen molecules are excited to the sigma singlet state by constant pumping fluence. For example, a typical PDT regimen using porfimer sodium as the photosensitizer achieves steady state in 10 μs. Equation 3 represents the case of a very short pumping pulse which is represented as a delta function times the number of photons in the pulse, as discussed in Niedre, M., et al. “Direct Near-Infrared Photoluminescence Detection of Singlet Oxygen Generated by Photodynamic Therapy in Cells In Vitro and Tissues In vivo;” and Patterson, M. S., et al. “Experimental Tests of the Feasibility of Singlet Oxygen Photoluminescence Monitoring in vivo During Photodynamic Therapy.”Using Equations 2 and 3, the concentration of delta singlet state oxygen molecules can be measured based on photoluminescence in the case of the steady state or a short pumping excitation.

The photoluminescence (L) can be calculated using Equation 4 (approximated by Equation 6):

$$\begin{matrix}L = {\int }_{V} \frac{{[}^{1} {\sum }_{g}^{+} ] {R}_{1.63}}{{τ}_{1.63}} d V \approx \frac{1}{{τ}_{1.63}} {[}^{1} {\sum }_{g}^{+} ] {\int }_{V} {R}_{1.63} d V & ( 4 ) \\ taking R as isotropic and {\int }_{V} {R}_{1.63} d V \approx 1 & ( 5 ) \\ L \approx \frac{1}{{τ}_{1.63}} {[}^{1} {\sum }_{g}^{+} ] & ( 6 )\end{matrix}$$

R_{1.63}=radiative spatial factor measuring signal variance at 1.63 eV over spatial positions

For illustrative purposes, R is taken as isotropic and unity. In practice R can vary with the particular photoluminescence detector configuration implemented. R can be set by calibration of the photoluminescence detector.

In an illustrative embodiment, the concentration of delta singlet state oxygen molecules can be calculated with Equations 7 or 9:

$$\begin{matrix}\begin{matrix}{[}^{1} {O}_{2} ] = \frac{{τ}_{1.63} \cdot L}{{I}_{\sum } {σ}_{Δ}} ( \frac{1}{{τ}_{1.63}} + \frac{1}{{τ}_{0.65}} ) & ( in steady state )\end{matrix} & ( 7 ) \\ = \frac{L}{{I}_{\sum } {σ}_{\sum }} ( 1 + \frac{{τ}_{1.63}}{{τ}_{0.65}} ) & ( 8 ) \\ \begin{matrix}{[}^{1} {O}_{2} ] = ( \frac{{τ}_{1.63}}{N {σ}_{Δ} \cdot exp ( \frac{- t}{{τ}_{1.63}} + \frac{- t}{{τ}_{0.63}} )} L ) & ( with I = N \cdot δ ( t ) )\end{matrix} & ( 9 )\end{matrix}$$

At steady state, Equation 7 can be used with absolute photoluminescence values (photon counts or integrations) to directly calculate the concentration of delta singlet state oxygen molecules. Equation 9 can be fitted over the time resolved photoluminescence to give the concentration of delta singlet state oxygen molecules at particular time points.

Equations 7 and 9 can be further simplified when the magnitude of the lifetimes in solution is known. The lifetime of the 1.63 eV transition (sigma singlet state to sigma triplet state) is likely to be approximately 1/3000 of the lifetime of the 0.65 eV transition (sigma singlet state to delta singlet state), as discussed in Weldon, D., et al. “Singlet Sigma: The ‘Other’ Singlet Oxygen in Solution,” Photochemistry and Photobiology (1999) 70: 369-379, which is incorporated herein by reference in its entirety. Equations 10 and 11 are simplified for a 3000/1 state lifetime differential:

For the case, 3000·τ_{1.63}≈τ_{0.65} in solution:

$$\begin{matrix}\begin{matrix}{[}^{1} {O}_{2} ] \approx \frac{L}{{I}_{\sum } {σ}_{Δ}} \cdot 3 \times {10}^{3} & ( in steady state )\end{matrix} & ( 10 ) \\ \begin{matrix}{[}^{1} {O}_{2} ] \approx \frac{{τ}_{1.63} \cdot L}{N {σ}_{Δ} \cdot exp ( \frac{- 3000 L}{{τ}_{1.63}} )} & ( with I = N \cdot δ ( t ) )\end{matrix} & ( 11 )\end{matrix}$$

In an illustrative embodiment, a typical therapeutic configuration includes a 0.65 eV, 100 mW/cm² light source, a 10⁻¹⁷ oxygen cross section, and 10¹⁰ μM delta singlet oxygen originating from a PDT including a 100 mW/cm² excitation of porfimer sodium (also known by the trade name Photofrin) at an initial concentration of 8.5 μM. Applying Equation 10:

$$\begin{matrix}L \approx \frac{{I}_{\sum } {σ}_{Δ} {[}^{1} {O}_{2} ]}{3 \times {10}^{3}} & ( 12 ) \\ {[}^{1} {O}_{2} ] = {\frac{6.022 \times {10}^{23} molecules \text{/} mol}{{10}^{3} {cm}^{3} \text{/} L} {[}^{1} {O}_{2} ]}_{M} & ( 13 ) \\ {I}_{\sum } = \frac{φ}{1.04 \times {10}^{- 19} J \text{/} photon} & ( 14 ) \\ L \approx \frac{6.022 \times {10}^{20} molecules \cdot L \text{/} {cm}^{3} \cdot mol}{1.04 \times {10}^{- 19} J \text{/} photon \cdot 3 \times {10}^{3}} {σ}_{Δ} {φ {[}^{1} {O}_{2} ]}_{M} & ( 15 ) \\ L \approx 1.93 \times {10}^{36} \frac{molecules \cdot L \cdot photons}{J \cdot {cm}^{3} \cdot mol} {σ}_{Δ} {φ {[}^{1} {O}_{2} ]}_{M} & ( 16 ) \\ & ( 17 )\end{matrix}$$

Definitions:

φ=input radiation fluence (at 0.65 eV), in W/cm²

[¹O₂]_{M}=delta singlet oxygen concentration, in molarity M=mols/L

σ_{Δ}=cross section of delta singlet for excitation to sigma singlet, in cm⁻²

For example, in a typical Photofrin case with therapeutic doses, steady-state is achieved in 10 μs, and for parameters:

$$\begin{matrix}φ = 100 mW \text{/} {cm}^{2} , {σ}_{Δ} = {10}^{- 17} , {[}^{1} {O}_{2} ] = {10}^{10} μM & ( 18 ) \\ L \approx 1.93 \times {10}^{36} \cdot {10}^{- 17} {cm}^{- 1} \cdot {10}^{2} \times {10}^{- 3} W \text{/} {cm}^{2} \cdot {10}^{- 10} μM \approx 1.93 \times {10}^{3} \frac{photons}{{cm}^{2} \cdot s} & ( 19 )\end{matrix}$$

Thus, in the typical therapeutic configuration, 1.63 eV photons are produced at a rate on the order of milliseconds in numbers that can be readily detected with commercial photon detectors, such as CCD cameras or bolometric detectors. Even if the transition time differential is higher than 3,000 for 1.63 eV, the system can still readily detect the 1.63 eV photons since a large number of photons are available for detection. The fluence (i.e., the W/m) of the 0.65 eV excitation energy may also be increased to produce a higher phosphorescence signal.

In another illustrative embodiment, in the case where the 0.65 eV excitation is a pulse or train of pulses, the photoluminescence can be measured in the time-domain (using time-resolved spectroscopic methods) and then fitted to Equation 10, with the parameters (except L) defined as free parameters.

For example, from Equation 11, the exponential decay of the photoluminescence signal has a half-life (t_{1/2}):

$$\begin{matrix}L \sim exp ( \frac{- 3000 t}{{τ}_{1.63}} ) & ( 20 ) \\ At 1 / 2 L , exp ( \frac{- 3000 {t}_{1 / 2}}{{τ}_{1.63}} ) = \frac{1}{2} & ( 21 ) \\ ( \frac{- 3000 {t}_{1 / 2}}{{τ}_{1.63}} ) = - ln 2 & ( 22 ) \\ {t}_{1 / 2} = \frac{{τ}_{1.63} \cdot ln 2}{3000} & ( 23 ) \\ {t}_{1 / 2} = 2.31 \times {10}^{- 4} secs \cdot {τ}_{1.63} . & ( 24 )\end{matrix}$$

Where the lifetime of the sigma singlet state to sigma triplet state transition is at least on the order of tens of nanoseconds, methods for measuring exponential decay can be used, such as: super fast femtosecond photon counting (femtoseconds), spectroscopic methods (nanoseconds), and commodity imaging equipment (hundreds of nanoseconds and up).

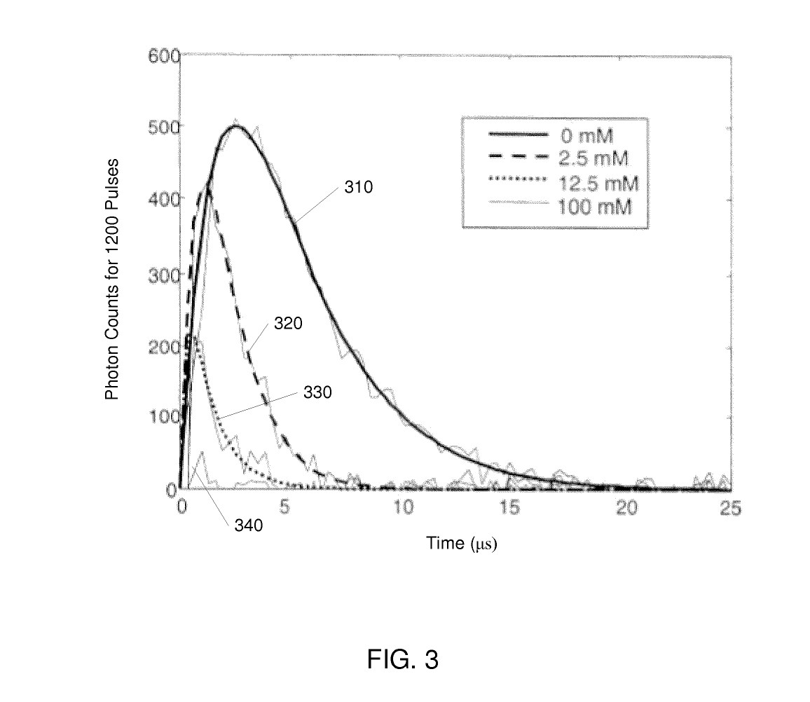

Referring to FIG. 3, a graph of photoluminescence levels in accordance with an illustrative embodiment is shown. FIG. 3 shows delta singlet photoluminescence (photon counts for 1200 pulses versus time (μs)) for various solutions of 6 μM tetra-sulfonated aluminum phthalocyanine (AlS₄Pc) in water with increasing sodium azide (NaN₃) concentration plotted from time-resolved measures at 1270 nm. Plot 310 shows a fitted curve for a concentration of 0 nM NaN₃. Plot 320 shows a fitted curve for a concentration of 2.5 nM NaN₃. Plot 330 shows a fitted curve for a concentration of 12.5 nM NaN₃. Plot 340 shows data for a concentration of 100 nM NaN₃. Photoluminescence can be determined for a pulse or pulses by curve-fitting the photon counts (i.e., plots 310, 320, 330, and 340) and integrating the fitted curve. Thus, the area under the fitted curve is approximately the total photon count.

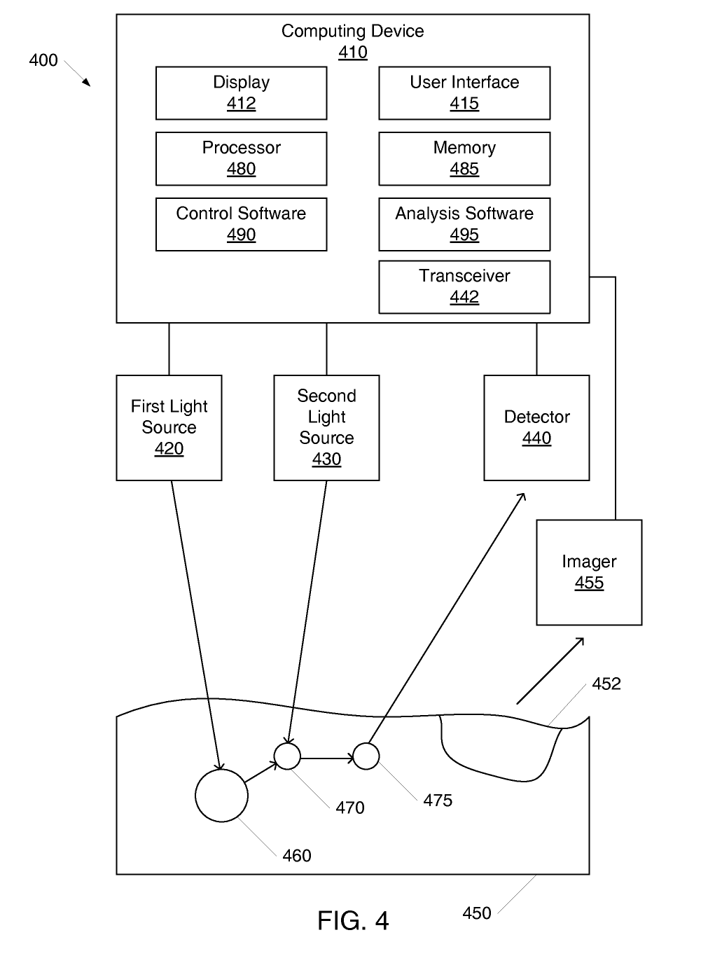

Referring to FIG. 4, a schematic of a delta singlet state oxygen dosimetry system 400 in accordance with an illustrative embodiment is shown. The delta singlet state oxygen dosimetry system 400 includes a computing device 410, a first light source 420, a second light source 430, and a detector 440. In alternative embodiments, system 400 may include additional, fewer, and/or different components. The first light source 420, the second light source 430, and the detector 440 can be positioned relative to a tissue 450. The tissue 450 can include a photosensitizer 460. The photosensitizer 460 can be injected into or applied to a subject of the tissue 450 (i.e., the patient). In one illustrative embodiment, the tissue 450 is cancerous. For example, the tissue 450 can be skin and the cancer can be melanoma. The tissue 450 can be from any animal, such as a human or non-human. The tissue 450 can be alive or dead, in vivo, ex vivo, or in vitro. Alternatively, the tissue 450 can be plant matter from a plant. The photosensitizer 460 can be, for example, but not limited to, porfimer sodium, aminolevulinic acid, methyl aminolevulinate, porphyrins, silicon phthalocyanine, m-tetrahydroxyphenylchlorin, mono-L-aspartyl chlorine, or any other photosensitizer.

The first light source 420 is configured to activate photosensitizer 460 thereby generating a delta singlet oxygen molecule 470, as described above. The first light source 420 can be operably (e.g., electrically) coupled to the computing device 410. For example, the first light source 420 can be wired or in wireless communication with the computing device 410. The first light source 420 can be a light source matched to the particular photosensitizer used. That is, the light source primarily generates light at a wavelength that activates the photosensitizer. The first light source 420 can operate in a continuous or pulsed mode, and can be coherent or non-coherent. For some photosensitizers, the first light source 420 can be any light source that produces mid-infrared photons. For example, the first light source 420 can be, but not limited to, a laser, a light emitting diode, a tungsten light energy, a mid-infrared GaSb heterostructure light emitting diode, a group III-V InGaAsP laser diode, an antimony laser diode, or a green field mid-infrared light source.

The second light source 430 is configured to generate a sigma singlet oxygen molecule 475 from the delta singlet oxygen molecule 470. The second light source 430 can be operably (e.g., electrically) coupled to the computing device 410. For example, the second light source 430 can be wired or in wireless communication with the computing device 410. The second light source 430 can operate in a continuous or pulsed mode. The delta singlet oxygen molecule 470 can damage the tissue 450 or a portion of the tissue 450 as described above. In one illustrative embodiment, the second light source 430 can be a 1907 nm light source that emits 0.65 eV photons. In another illustrative embodiment, the second light source 430 has a wavelength range of 1902 nm to 1912 nm. The second light source 430 can be, for example, a tungsten light source, mid-infrared GaSb heterostructure LEDs, group III-V InGaAsP or antimony GaInAsSb laser diodes, or other “green field” mid-IR sources. Alternatively, the first light source 420 and the second light source 430 can be the same light source.

The detector 440 can be configured to detect an emission of the sigma singlet oxygen molecule 475 decaying to the ground state, as described above. The detector 440 can be operably (e.g., electrically) coupled to the computing device 410. For example, the detector 440 can be wired or in wireless communication with the computing device 410. In one illustrative embodiment, the detector 440 can be configured to detect 1.63 eV photons (i.e., 762 nm light). In another illustrative embodiment, the detector 440 can detect wavelengths in a range from 757 nm to 767 nm. The detector 440 can be, for example, but not limited to, a charge-coupled device, a camera, a photodiode, a bolometer, a thermopile, or any other quantitative photon detection device.

The computing device 410 can be a circuit, a desktop computer, a laptop computer, a cloud computing client, a hand-held computing device, or other type of computing device known to those of skill in the art. The computing device 410 includes one or more of, a memory 485, control software 490, analysis software 495, a processor 480, a display 412, transceiver 442, and a user interface 415. In alternative embodiments, the computing device 410 may include fewer, additional, and/or different components. The memory 485, which can be any type of permanent or removable computer memory known to those of skill in the art, can be a computer-readable storage medium. The memory 485 is configured to store one or more of the control software 490, the analysis software 495, an application configured to run the control software 490 and the analysis software 495, captured data, and/or other information and applications as known to those of skill in the art. The transceiver 442 of the computing device 410 can be used to receive and/or transmit information through a wired or wireless network as known to those of skill in the art. The transceiver 442, which can include a receiver and/or a transmitter, can be a modem or other communication component known to those of skill in the art.

The analysis software 495 is configured to analyze captured photon data from the detector 440 and to determine the dosimetry of the delta singlet oxygen. The captured data can be received by the computing device 410 through a wired connection such as a USB cable and/or through a wireless connection, depending on the embodiment. The captured data may include the photon data before, during, and after application of the photosensitizer 460. The analysis software 495, which can be implemented as computer-readable instructions configured to be stored on the memory 485, can analyze the captured data to determine a concentration of sigma singlet oxygen and delta singlet oxygen, as described above.

In one embodiment, the analysis software 495 can include a computer program and/or an application configured to execute the program such as Matlab. Alternatively, other programming languages and/or applications known to those of skill in the art can be used. In one embodiment, the analysis software 495 can be a dedicated standalone application. The processor 480, which can be in electrical communication with each of the components of the computing device 410, can be used to run the application and to execute the instructions of the analysis software 495. Any type of computer processor(s) known to those of skill in the art may be used.

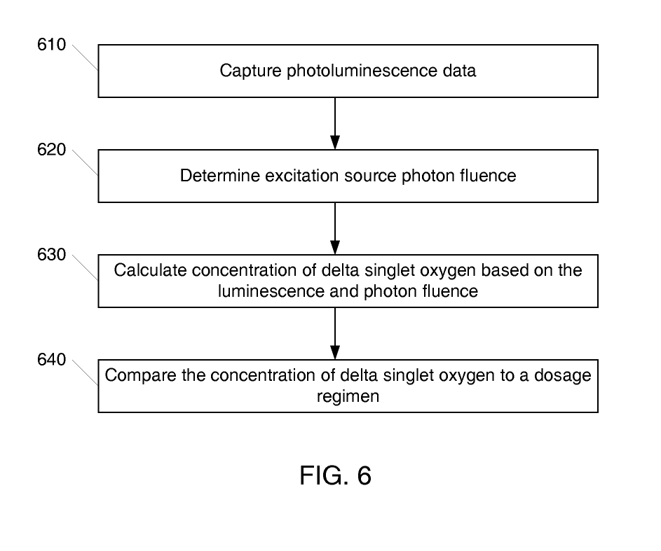

Referring to FIG. 6, a flow diagram of analysis software 495 of FIG. 4 in accordance with an illustrative embodiment is shown. In alternative embodiments, fewer, additional, and/or different operations may be performed. In one illustrative embodiment, the analysis software 495 can solve Equation 10, from above, based on photoluminescence data from the detector 440 and the fluence of the second light source 430. In an operation 610, photoluminescence data can be captured, for example, by detector 440. Optionally, the data from the detector 440 can be adjusted to account for red shifting of wavelengths by the tissue 450, as discussed above.

In an operation 620, the photon fluence of the excitation source is determined. For example, the photon fluence of the second light source 430 can be determined by a detector or by estimating the photon fluence based on the power delivered to the second light source 430.

In an operation 630, the concentration of delta singlet oxygen can be calculated based on the luminescence and photon fluence photon fluence of the excitation source. The photoluminescence data and photon fluence can be used to solve Equation 10, from above. The result is the approximate concentration of delta singlet oxygen. Optionally, data from other sensors can be considered. In addition, a user can enter into the computing device 410 through the user interface 415 other information or change variables of Equation 10 and its related equations.

In another illustrative embodiment, the analysis software 495 can solve Equation 11, from above, based on photoluminescence data from the detector 440, the fluence of the second light source 430, and, optionally, the fluence of the first light source 420. In another illustrative embodiment, the analysis software 495 can adapt a kinetic differential equation such as Equation 1 for a particular situation, as described above.

In an operation 640, after determining the concentration of delta singlet oxygen, the analysis software 495 can compare the concentration of delta singlet oxygen to a dosage regimen. In one illustrative embodiment, the dosage regimen can be a photodynamic therapy dosage regimen for porfimer sodium, as is well known in the art. The dosimetry can be determined using curve fitting methods, analyses of exponential decay of photoluminescence, Fourier transform spectroscopy, and other time resolved methods. If the current concentration of delta singlet oxygen is below a recommended dosage, the analysis software 495 can request the control software 490 to increase the concentration of delta singlet oxygen. If the current concentration of delta singlet oxygen is above the recommended dosage, the analysis software 495 can request the control software 490 to decrease the concentration of delta singlet oxygen.

In one illustrative embodiment, a curve is fitted to the emitted photon counts from sigma singlet decay measured over a period of time. After adjusting for the efficiency of excitation from delta singlet to sigma singlet oxygen, the total delta singlet population over the time period can be determined by integrating the fitted curve over the time period. The result is the dose (or concentration) of delta singlet oxygen. In general, time-resolved methods measure the exponential decay of the sigma singlet oxygen and, based on curve fitting methods, determine the total amount of the sigma singlet oxygen. For example, assuming the curve is exponential, the decay can be modeled as a tail thereby accounting for the full distribution of sigma singlet oxygen.

Referring again to FIG. 4, the control software 490 is configured to control the first light source 420 and the second light source 430. The first light source 420 and the second light source 430 can be communicatively coupled to the computing device 410 through a wired connection such as a USB cable and/or through a wireless connection, depending on the embodiment. The control software 490, which can be implemented as computer-readable instructions configured to be stored on the memory 485, can control the wavelength and/or power (fluence) of the first light source 420 and the second light source 430.

In one illustrative embodiment, the control software 490 can include a computer program and/or an application configured to execute the program such as Windows available from Microsoft Corp., Redmond, Wash. Alternatively, other programming languages and/or applications known to those of skill in the art can be used. In one embodiment, the control software 490 can be a dedicated standalone application. The processor 480, which can be in electrical communication with each of the components of the computing device 410, can be used to run the application and to execute the instructions of the control software 490. Any type of computer processor(s) known to those of skill in the art may be used.

In one illustrative embodiment, the control software 490 can set the power (fluence) of the first light source 420 and the second light source 430. For example, the power of the first light source 420 and the second light source 430 can be controlled by controlling the power available. Alternatively, the control software 490 can provide a power reference to the first light source 420 and the second light source 430 such as an analog output or a digital value, for example, via a serial port. In another illustrative embodiment, the control software 490 can control the waveform of the first light source 420 and the second light source 430. For example, the control software 490 can command the first light source 420 or the second light source 430 to pulse or generate a steady state fluence.

In one illustrative embodiment, the control software 490 can set the wavelength of the first light source 420 and the second light source 430. For example, a wavelength of the first light source 420 can be matched to the particular photosensitizer used. For example, a wavelength of the second light source 430 can be 1907 nm. In one illustrative embodiment, the wavelength of the first light source 420 and the wavelength of the second light source 430 can be adjusted to account for red shifting of wavelengths by the tissue 450. In another illustrative embodiment, the second light source 430 can be adjusted in a wavelength range of 1902 nm to 1912 nm.

In another illustrative embodiment, the control software 490 can control the first light source 420 and the second light source 430 to target a portion of tissue 452 of the tissue 450. For example, the portion of tissue 452 can be cancerous. An imager 455 can be communicatively coupled to computing device 410. The imager 455 can be, for example, but not limited to, a camera, a tomograph, a computerized axial tomography scanner, a magnetic resonance imager, etc. The imager 455 can detect cancer of the tissue 450 and define the portion of tissue 452 as a treatment area. The imager 455 can provide targeting information such as a location, type, material, density, etc. of the portion of tissue 452 to the computing device 410. The control software 490 can use the targeting information to direct and control the first light source 420 and the second light source 430, as described above.

In one illustrative embodiment, the first light source 420 and the second light source 430 can produce beams of light. The control software 490 can direct a beam of the first light source 420 and a beam of the second light source 430 to scan throughout a volume of the portion of tissue 452 based on the targeting information. The control software 490 can use the results of the analysis software 495 to determine if a proper dosage has been administered. If the proper dosage has not been administered to a particular area of the portion of tissue 452, the control software 490 can direct the first light source 420 and the second light source 430 to rescan the particular area of the portion of tissue 452. Thus, in one illustrative embodiment, only the treatment area is exposed to the photodynamic therapy. Advantageously, by scanning a focused beam of the second light source 430, the dosage of the immediate area of treatment can be determined instead of an aggregate dosage for a large area.

The display 412 of the computing device 410 can be used to display one or more images of data from the detector 440, a user interface window through which a user can control detector 440, the first light source 420, the second light source 430, the analysis software 495, the control software 490, etc., plots illustrating the dosage and dosage regiment, etc. The display 412 can be a liquid crystal display, a cathode ray tube display, or other type of display known to those of skill in the art. The user interface 415 allows a user to interact with computing device 410 and to enter information into the user interface window. The user interface 415 can include a mouse, a keyboard, a touch screen, a touch pad, etc. The user can use the user interface 415 to control the on/off status of the detector 440, the first light source 420, the second light source 430, etc.

In the embodiment illustrated with reference to FIG. 4, the computing device 410, the first light source 420, the second light source 430, and the detector 440 are illustrated as separate components that are combined to form the delta singlet state oxygen dosimetry system 400. In an alternative embodiment, any or all of the components of delta singlet state oxygen dosimetry system 400 may be integrated into a dedicated stand-alone apparatus that has the functionality described with reference to FIG. 4.

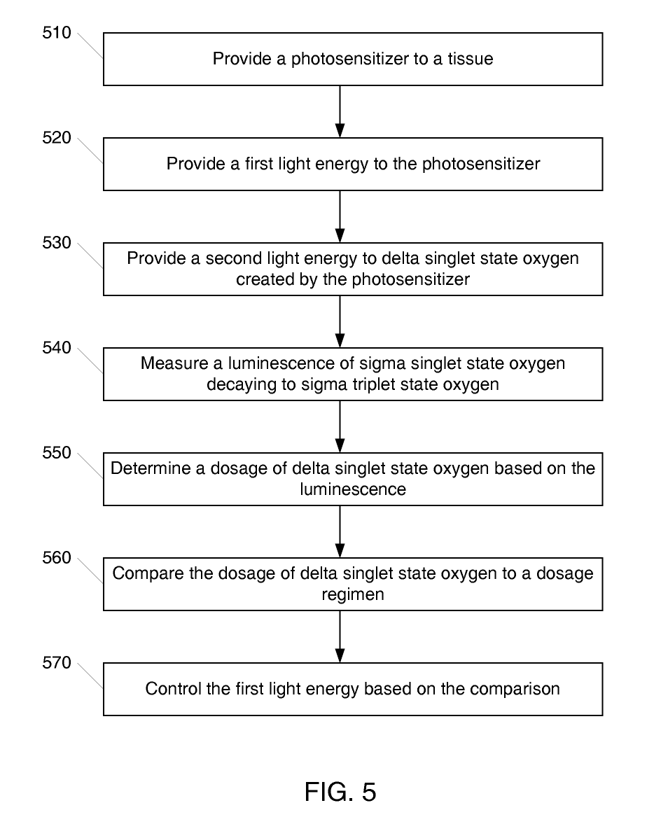

Referring to FIG. 5, a flow diagram illustrating delta singlet state oxygen dosimetry operations performed in accordance with an illustrative embodiment is shown. In alternative embodiments, fewer, additional, and/or different operations may be performed. In an illustrative embodiment, the delta singlet state oxygen dosimetry operations can be performed by a delta singlet state oxygen dosimetry system described with reference to FIG. 4.

In an operation 510, a photosensitizer can be provided to a tissue. The photosensitizer can be porfimer sodium, etc. as described above. The photosensitizer can be applied in any appropriate manner, including but not limited to, injected, administered orally, or applied topically. The tissue can be human, non-human, cancerous, etc., as described above.

In an operation 520, a first light energy can be provided to the photosensitizer. In one illustrative embodiment, the first light energy is configured to cause the photosensitizer to create delta singlet state oxygen in the tissue. In one illustrative embodiment, the first light energy can be adjusted to compensate for red shifting caused by the tissue.

In an operation 530, a second light energy is provided to the delta singlet state oxygen created by the photosensitizer. The second light energy can cause the delta singlet state oxygen to excite to sigma singlet state oxygen. The second light energy can be for example 1907 nm light, or a range of light from about 1902 nm to about 1912 nm. The second light energy can be generated by a light source such as a tungsten light source, etc., as described above. In one illustrative embodiment, the second light energy can be adjusted to compensate for red shifting caused by the tissue.

In an operation 540, a photoluminescence of sigma singlet state oxygen decaying to sigma triplet state oxygen can be measured, for example, by a detector. As described above, when the sigma singlet state oxygen decays to sigma triplet state oxygen, the oxygen can emit a 760 nm photon. In one illustrative embodiment, the detector can detect 760 nm light, or a range of light from about 755 nm to 765 nm. The detector can be, for example, a charge-coupled device, etc., as described above. In one illustrative embodiment, the detector can be adjusted to compensate for red shifting caused by the tissue.

In an operation 550, a dosage of delta singlet state oxygen can be determined based on the photoluminescence detected in operation 540. In one illustrative embodiment, the photoluminescence can be used to solve a kinetic differential equation to determine a concentration of delta singlet state oxygen, as described above. For example, Equation 10 can be used to solve for the concentration of delta singlet state oxygen where the second light energy is in the steady state, as described above. For example, Equation 11 can be used to solve for the concentration of delta singlet state oxygen where the second light energy is pulsed, as described above. The concentration of delta singlet state oxygen can be correlated to a dosage of delta singlet state oxygen in relation to a photodynamic therapy dosage regimen.

In an operation 560, the dosage of delta singlet state oxygen can be compared a photodynamic therapy dosage regimen. If the current concentration of delta singlet oxygen is below a recommended dosage, the concentration of delta singlet oxygen can be increased. If the current concentration of delta singlet oxygen is above the recommended dosage, the concentration of delta singlet oxygen can be decreased.

In an operation 570, the first light energy can be controlled based on the comparison of the dosage to the regimen as in operation 560. When the intensity of the first light energy is increased the amount of delta singlet oxygen created is increased because the number of photosensitizer molecules that are activated increases. Conversely, when the intensity of the first light energy is decreased the amount of delta singlet oxygen created is decreased because the number of photosensitizer molecules that are activated decreases. In another illustrative embodiment, when the dosage of delta singlet state oxygen is low, more photosensitizer can be provided to the tissue in order to increase the probability that the first light energy will strike a photosensitizer molecule.

Advantageously, the delta singlet state oxygen dosimetry system and method can use off-the-shelf and commercially available excitation sources and detectors. Advantageously, since the excitation energy (0.65 eV) can be different than the detection energy (1.63 eV), background effects such as auto-fluorescence, present in direct delta singlet state luminescent detection, are avoided. Advantageously, the determination of the concentration of delta singlet state oxygen can be entirely non-invasive and does not require administration of additional in vivo components, as in chemiluminescent detection. Advantageously, the determination of the concentration of delta singlet state oxygen does not depend on a particular photosensitizer, unlike methods such as fluorescence monitoring photosensitizers, photobleaching of the photosensitizers, etc. Advantageously, the delta singlet state oxygen dosimetry system and method can determine dosimetry using a stable, observable effect, whereas other measures may change based on unknown parameters (such as biological parameters). Advantageously, the determination of the concentration of delta singlet state oxygen can have a direct correlation to therapeutic moiety. By measuring the actual therapeutic agent (delta singlet oxygen), dosimetry overcomes the patient-to-patient variability in photosensitizer pharmacokinetics, variance in tissue optical properties, differences in tissue oxygenation, and interdependence of all these factors.

One or more flow diagrams may have been used herein. The use of flow diagrams is not meant to be limiting with respect to the order of operations performed. The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

What is claimed is:

1. A method comprising:
measuring a photoluminescence of sigma singlet state oxygen decaying to triplet state oxygen; and
determining a dosage of delta singlet state oxygen based on the photoluminescence.

2. The method of claim 1, further comprising:
providing a light energy to a photosensitizer in a tissue wherein the light energy is configured to excite delta singlet state oxygen to sigma singlet state oxygen.

3. The method of claim 2, wherein the light energy is generated by at least one of a laser, a light emitting diode, a tungsten light energy, a mid-infrared GaSb heterostructure light emitting diode, a group III-V InGaAsP laser diode, an antimony laser diode, a green field mid-infrared light source, a mid-infrared coherent light source, and a mid-infrared non-coherent light source.

4. The method of claim 2, further comprising controlling the dosage of delta singlet state oxygen by controlling a light source configured to generate the delta singlet state oxygen via a photosensitizer in the course of a photodynamic therapeutic treatment.

5. The method of claim 1, further comprising providing a photosensitizer configured to generate the delta singlet state oxygen, wherein the photosensitizer is at least one of porfimer sodium, aminolevulinic acid, methyl aminolevulinate, porphyrins, silicon phthalocyanine, m-tetrahydroxyphenylchlorin, or mono-L-aspartyl chlorine.

6. The method of claim 1, wherein said measuring the photoluminescence comprises providing a detector, wherein the detector is at least one of a charge-coupled device, a camera, a photodiode, a bolometer, and a thermopile.

7. The method of claim 1, wherein said determining the dosage of delta singlet state oxygen based on the photoluminescence comprises determining a concentration of delta singlet state oxygen where the concentration of delta singlet state oxygen is determined by:
$${[}^{1} {O}_{2} ] = \frac{{τ}_{1.63} \cdot L}{{I}_{\sum } {σ}_{Δ}} ( \frac{1}{{τ}_{1.63}} + \frac{1}{{τ}_{0.65}} )$$
where [¹O₂] is the concentration of delta singlet state oxygen, τ_{1.63} is the lifetime transition of sigma singlet state oxygen decaying to triplet state oxygen, τ_{0.65} is the lifetime transition of delta singlet state oxygen to sigma singlet state oxygen, σ_{Δ} is the cross section of delta singlet state oxygen for excitation to sigma singlet state oxygen, I_{Σ} is the excitation photon fluence of a light energy, and L is the photoluminescence.

8. The method of claim 1, wherein said determining the dosage of delta singlet state oxygen based on the photoluminescence comprises determining a concentration of delta singlet state oxygen where the concentration of delta singlet state oxygen is determined by:
$${[}^{1} {O}_{2} ] = ( \frac{{τ}_{1.63}}{N {σ}_{Δ} \cdot exp ( \frac{- t}{{τ}_{1.63}} + \frac{- t}{{τ}_{0.65}} )} L )$$
where [¹O₂] is the concentration of delta singlet state oxygen, τ_{1.63} is the lifetime transition of sigma singlet state oxygen decaying to triplet state oxygen, τ_{0.65} is the lifetime transition of delta singlet state oxygen to sigma singlet state oxygen, σ_{Δ} is the cross section of delta singlet state oxygen for excitation to sigma singlet state oxygen, N is the number of photons from a light energy, and L is the photoluminescence.

9. An apparatus comprising:
a detector configured to measure a photoluminescence of sigma singlet state oxygen decaying to triplet state oxygen; and
a module configured to determine a dosage of delta singlet state oxygen based on the photoluminescence.

10. The apparatus of claim 9, further comprising a light source configured to excite delta singlet state oxygen to sigma singlet state oxygen in vivo in a tissue comprising a photosensitizer.

11. The apparatus of claim 10, wherein the light source is at least one of a laser, a light emitting diode, a tungsten light energy, a mid-infrared GaSb heterostructure light emitting diode, a group III-V InGaAsP laser diode, an antimony laser diode, a green field mid-infrared light source, a mid-infrared coherent light source, and a mid-infrared non-coherent light source.

12. The apparatus of claim 10, further comprising a second light source wherein the module is configured to control the dosage of delta singlet state oxygen by controlling the second light source, wherein light energy of the second light source is configured to generate the delta singlet state oxygen via the photosensitizer in the course of a photodynamic therapeutic treatment.

13. The apparatus of claim 12, wherein the photosensitizer is at least one of porfimer sodium, aminolevulinic acid, methyl aminolevulinate, porphyrins, silicon phthalocyanine, m-tetrahydroxyphenylchlorin, and mono-L-aspartyl chlorine.

14. The apparatus of claim 9, wherein the detector is at least one of a charge-coupled device, a camera, a photodiode, a bolometer, and a thermopile.

15. The apparatus of claim 9, wherein the module is configured to determine a concentration of delta singlet state oxygen where the concentration of delta singlet state oxygen is proportional to the photoluminescence of sigma singlet state oxygen decaying to triplet state oxygen and an excitation input configured to excite delta singlet state oxygen to sigma singlet state oxygen.
